(19) United States
(12) Patent Application Publication
(10) Pub. No.: US 2022/0040158 A1
(43) Pub. Date: Feb. 10, 2022

(54) AZD0328 DOSAGE REGIME FOR TREATING COGNITIVE IMPAIRMENT

(71) Applicant: ASTRAZENECA AB, SODERTALJE (SE)

(72) Inventor: DAVID JOHN HAYES, WILIMINGTON, DE (US)

(21) Appl. No.: 17/278,916

(22) PCT Filed: Sep. 23, 2019
(86) PCT No.: PCT/EP2019/075569

§ 371 Date: Mar. 23, 2021

Related U.S. Application Data
(60) Provisional application No. 62/735,494, filed on Sep. 24, 2018.
(60) Provisional application No. 62/741,103, filed on Oct. 4, 2018.

(51) Int. Cl. A61K 31/439; C07D 491/22; A61P 25/28

(52) U.S. Cl. CPC A61K 31/439; A61P 25/28; C07D 491/22

(57) ABSTRACT

The nicotinic acetylcholine receptor alpha 7 (□7 nAChR) agonist ((3R)-Spiro[1-azabicyclo[2.2.2]octane-3,2′(3′H)-furo[2,3-b]pyridine, or a pharmaceutically acceptable salt thereof, for use in the treatment or prophylaxis of mild cognitive impairment (MCI) in Parkinson's disease, wherein the compound is administered in a unit dose of from 0.25 mg to 0.50 mg twice daily. Methods of treatment or prophylaxis of MCI and kits for use in the such methods are also provided.

The present specification relates to the nicotinic acetylcholine receptor alpha 7 (α7 nAChR) agonist (3R)-spiro[1-azabicyclo[2.2.2]octane-3,2′(3′H)-furo[2,3-b]pyridine (AZD0328) for use in the treatment of mild cognitive impairment (MCI) in patients suffering from Parkinson's disease (PD) wherein the compound is administered at a specified dose and dosing frequency.

Nicotinic acetylcholine receptor alpha 7 (α7 nAChRs or α7 receptors) are ligand-gated ion channels implicated in synaptic heteroreceptor modulation of major neurotransmitter systems, synaptic plasticity, and learning and memory. Due to these functions, targeting of α7 nAChR has long been considered a promising potential therapeutic approach for the treatment of diseases which result in cognitive impairment (see e.g. Lewis et al. (2017) “Alpha-7 nicotinic agonists for cognitive deficits in neuropsychiatric disorders: A translational meta-analysis of rodent and human studies” Prog Neuropsychopharmacol Biol Psychiatry. 2017 Apr. 3; 75: 45-53). Agonism of α7 nAChR has thus been considered as a potential therapeutic strategy for the treatment of psychiatric diseases such as schizophrenia and neurodegenerative diseases such as Alzheimer's disease (AD) for a number of years.

In the case of schizophrenia, it has been widely postulated that direct-acting agonists of various cholinergic receptors including α7 nAChRs, could normalize GABAergic, glutamergic, or dopaminergic function in the dorsolateral prefrontal cortex (dIPFC), dysfunction of which has been consistently associated with cognitive deficits in this condition. Clinical evaluation of the potent α7 nAChR agonist AZD0328 in schizophrenia patients at doses up to 0.675 mg (plasma levels at ca 5×IC₅₀) did not show a statistically significant improvement in cognition and trials were subsequently terminated. To date, despite numerous trials, no α7 nAChR agonist has been approved for use in the treatment of schizophrenia.

Alzheimer's disease (AD) is the most common, and invariably fatal, neurodegenerative disease and is characterized by progressive impairment of memory, learning abilities, object recognition, disorientation, and decline in language function. Neurodegeneration in AD patients is characterized by progressive loss of neurons in the basal forebrain that synthesize and release the neurotransmitter acetylcholine (ACh). Acetylcholinesterase (AChE) terminates cholinergic neurotransmission by hydrolysing acetylcholine. Treatment with acetylcholinesterase inhibitors (AChEI) provides AD patients with modest symptomatic improvement in cognitive function, most likely by prolonging cholinergic neurotransmission. It has long been postulated that direct-acting agonists of various cholinergic receptors, including α7 nAChR, might restore lost cholinergic receptor signaling and deliver improvements over AChEI. Clinical studies with α7 nAChR agonists in AD patients have not yet delivered new therapeutic agents for the treatment of this condition.

After AD, Parkinson's disease (PD) is the second most common neurodegenerative disease. Parkinson's disease affects approximately 1% of people over 65, with 2.2 million people suffering from the disease in the USA and Europe. Around, 60,000 people in the USA are newly diagnosed with Parkinson's disease each year (e.g. see de Lau et al. Epidemiology of Parkinson's disease. Lancet Neurol. (2006), 5 525-535 and Olesen et al. (2012) “The economic cost of brain disorders in Europe” Eur J Neurol 19, 155-162).

Some 25% of Parkinson's disease patients develop dementia. Of the non-demented cohort of PD patients about 20% exhibit a mild cognitive impairment (MCI) that results in a reduced quality of life, caregiver stress, and higher health-related costs. In addition, patients with MCI progress to dementia over a shorter period than those without MCI (e.g. see Aarsland et al 2010 “Mild cognitive impairment in Parkinson's disease a multicentre pooled analysis” Neurology 75, 1062-1069). Therefore, treatment of MCI in PD is a high unmet medical need.

As with schizophrenia and AD, a body of literature supports the role of nicotinic receptors in PD and cognition. The cholinergic nicotinic receptors expressed in the highest concentrations in the human CNS are the α4β2 and α7 receptors. Nicotinic receptors affect cholinergic, dopaminergic, glutamatergic and other systems known to be involved in cognitive decline in PD, and of particular relevance is their dopamine-releasing effect in the ventral tegmental area which is related to attentional and executive deficits in Parkinson's disease. The established link between smoking and reduced risk of PD also underlines the relevance of the nicotinic system in this condition. Importantly, α7 nicotinic receptor changes are particularly pronounced in Parkinson's disease more, so than in other neuropsychiatric diseases, and these changes are associated with key clinical features. Although clinical trials of α7 nAChR agonists have not delivered the hoped for improvement in levodopa-induced dyskinesia, a recent study with the α7 receptor agonist AQW051 has indicated that improvements in cognitive function can be realised. (Trenkwalder et al, Movement Disorders, 31(7), 2016, p 1049-1054). Despite this promising result, development of AQW051 was abandoned. No α7 nAChR agonist has been approved for clinical use for the treatment of PD or allieviation of its symptoms.

It is an object of the present specification to provide a therapeutic approach for the treatment of mild cognitive impairment in Parkinson's disease patients that involves administration of AZD0328 in a specific dose range and frequency of dosing.

(3R)-Spiro[1-azabicyclo[2.2.2]octane-3,2′(3′H)-furo[2,3-b]pyridine, also known as AZD0328, was first described in WO99/03859 and is a potent, full agonist of the human α7 nAChR (binding IC₅₀ of 3 nM; activation of whole cell current IC₅₀ of 2.9 μM; intrinsic activity=101% compared with acetylcholine). AZD0328 is ca 20-fold selective to the α1β1γδ nAChR, and 1000-fold selective to other nicotinic receptors and a panel of other targets (as determined by inhibition of radioligand binding).

In preclinical studies, oral administration of AZD0328 was found to significantly improve operant conditioning and long-term potentiation in rats (WO2008/115139). In Rhesus monkeys, spatial working memory was enhanced by doses of AZD0328 above 0.001 mg/kg (plasma compound levels of ca 0.2 times the whole cell current IC₅₀). Despite these promising preclinical findings, a clinical evaluation of AZD0328 in schizophrenia patients at doses up to 0.675 mg (corresponding to plasma levels of ca 5×IC₅₀) did not show a statistically significant improvement in cognition.

From analysis of the published data for the α7 nAChR agonist AQW051, and the promise that this molecule showed in delivering an improvement in cognitive function in certain PD patients, we conceived that administering AZD0328, a more potent α7 nAChR agonist, at a suitable dose and frequency could provide a new and advantageous approach for the treatment of PD patients with mild cognitive impairment. Intriguingly, should improvements in cognitive function that we had observed in rodents translate to humans there would seem to be potential for a disease modulating therapeutic intervention in non-demented Parkinson's disease patients with MCI.

Accordingly, in a first aspect the present specification provides (3R)-spiro[1-azabicyclo[2.2.2]octane-3,2′(3′H)-furo[2,3-b]pyridine (AZD0328), or a pharmaceutically acceptable salt thereof, for use in the treatment or prophylaxis of mild cognitive impairment in Parkinson's disease, wherein the compound is administered in a unit dose of from 0.25 mg to 0.50 mg twice daily.

In a further aspect, the specification provides a method of treatment or prophylaxis of mild cognitive impairment in Parkinson's disease, wherein the method involves administration of (3R)-spiro[1-azabicyclo[2.2.2]octane-3,2′(3′H)-furo[2,3-b]pyridine, or a pharmaceutically acceptable salt thereof, to a patient in need thereof at in a unit dose of from 0.25 mg to 0.50 mg twice daily.

In a further aspect, the specification provides (3R)-Spiro[1-azabicyclo[2.2.2]octane-3,2′(3′H)-furo[2,3-b]pyridine, or a pharmaceutically acceptable salt thereof, for use in the manufacture of a medicament for the treatment or prophylaxis of mild cognitive impairment in Parkinson's disease, wherein the medicament is to be administered on a twice daily basis and is in a unit dose of from 0.25 mg to 0.50 mg.

In a further aspect, the specification provides (3R)-Spiro[1-azabicyclo[2.2.2]octane-3,2′(3′H)-furo[2,3-b]pyridine, or a pharmaceutically acceptable salt thereof, for use in the treatment or prophylaxis of mild cognitive impairment in Parkinson's disease, wherein the compound is dosed in an amount and frequency to maintain a plasma concentration below 25 nM and wherein the plasma concentration is maintain at or above 5 nM for at least 20 h per day.

In a further aspect, the specification provides a kit containing (3R)-Spiro[1-azabicyclo[2.2.2]octane-3,2′(3′H)-furo[2,3-b]pyridine, or a pharmaceutically acceptable salt thereof, together with instructions for the use thereof for treatment or or prophylaxis of mild cognitive impairment in Parkinson's disease, optionally wherein the compound is provided as unit doses containing from 0.25 mg to 0.50 mg.

So that the claimed invention may be further understood, the specification herein refers to the following figures:

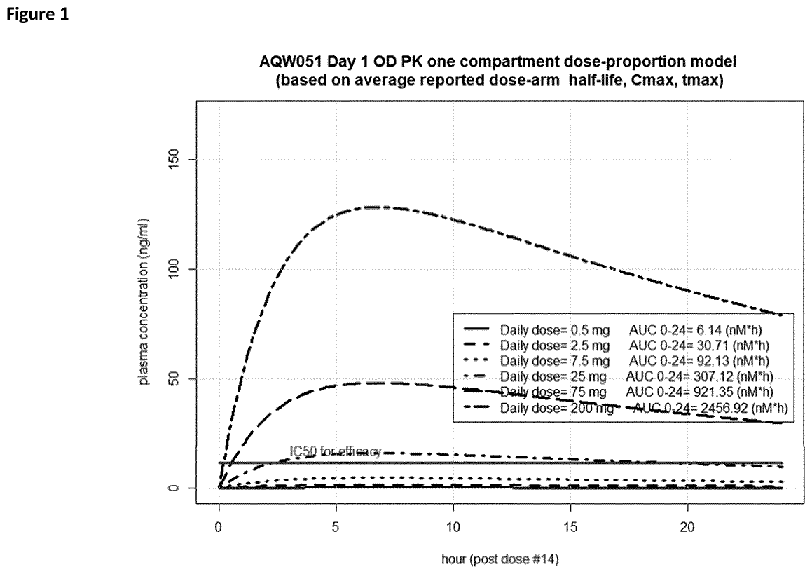

FIG. 1 AQW051 concentration profile for first day of dosing, including model-based AUC estimates;

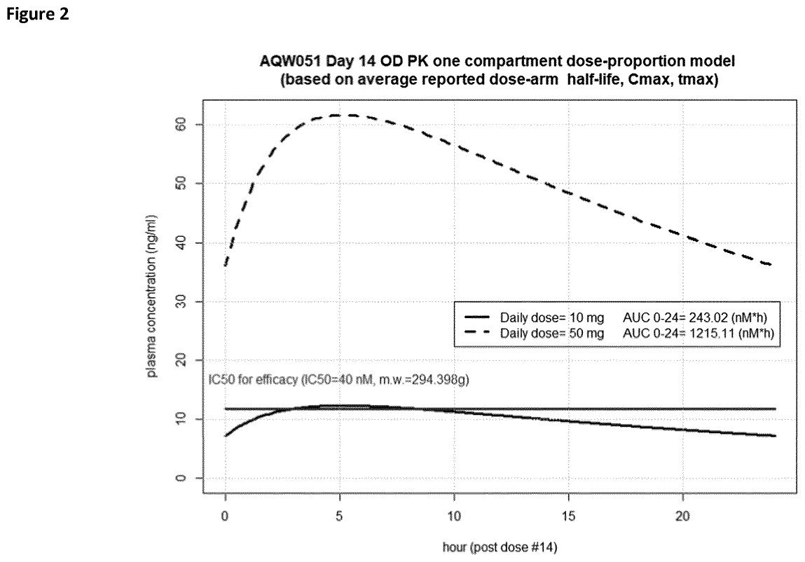

FIG. 2 AQW051 concentration profile for day 14 of once-per-day dosing, including model-based AUC estimates;

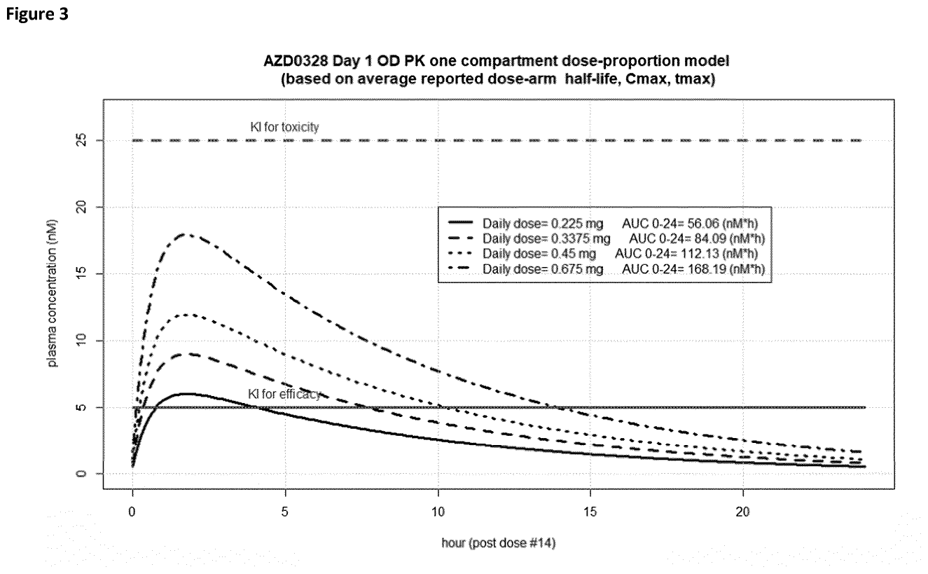

FIG. 3 AZD0328 concentration profile for first day of dosing, including model-based AUC estimates;

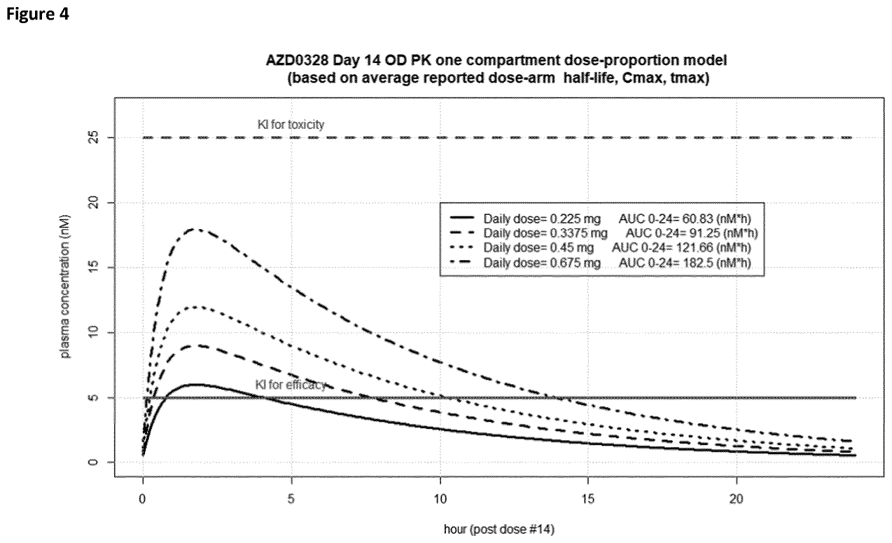

FIG. 4 AZD0328 concentration profile for day 14 of once-per-day dosing, including model-based AUC estimates; and

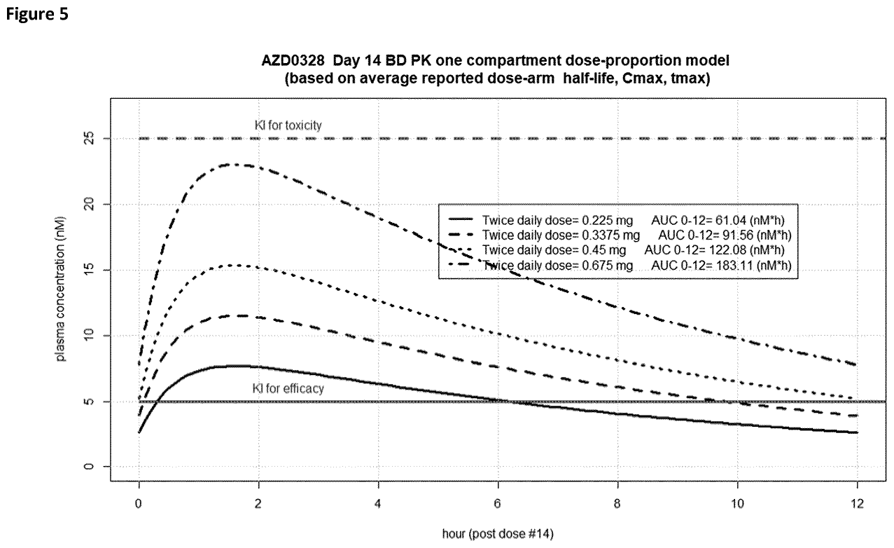

FIG. 5 AZD0328 concentration profile for day 14 of twice-per-day dosing, including model-based AUC estimates.

Mild cognitive impairment (MCI) is common in nondemented Parkinson's Disease patients and may be a harbinger of dementia and is predictive of the development of dementia over the long term. Diagnositic criteria for the identification of MCI in PD patients have been developed by the Movement Disoorder Society Task Force (Litvan et al, Mov Disord. (2012), 27(3), 349-356) and may be applied for selection of patients for treatment with AZD0328. The skilled person will therefore be able to select non-demented PD patients suffering from, or susceptible, to develop MCI based on these guidelines.

The results from a clinical trial performed with AQW051 in PD patients provide information on the plasma concentration of the compound achieved in Parkinson's disease patients on 10 mg, and 50 mg, once daily dosing. Although both treatment arms delivered an improvement in cognitive state, only the higher dose delivered a statistically significant improvement in MCI (effect size 0.5, P=0.024 cf effect size 0.4, P=0.073 in the lower dose cohort). The activity of AQW051 as an α7 nAChR agonist (EC₅₀ 40 nM) is known from the literature (e.g. see Feuerbach et al. (2014) “AQW051, a novel, potent and selective α7 nicotinic ACh receptor partial agonist: pharmacological characterization and phase I evaluation” British Journal of Pharmacology 172, 1292-1304 and Trenkwalder et al. “A Placebo-Controlled Trial of AQW051 in Patients With Moderate to Severe Levodopa-Induced Dyskinesia” Movement Disorders 31, 1049-1054).

We thus set out to use summary cohort-level pharmacokinetic (PK) statistics for AQW051 (Feuerbach et al, ibid) and our own unpublished results for AZD0328 (A Phase I, Single-Centre, Randomized, Double-Blind, Placebo-Controlled, Parallel Group Study to Assess the Safety, Tolerability and Pharmacokinetics of AZD0328 in Healthy Volunteers after Oral Single Ascending Doses, Study Code D0190C00005, Ed. 1, 8 Apr. 2008) to establish a safe an efficacious dose range and regime for AZD0328 in Parkinson's disease patients.

In more detail, a single compartment oral dosing model with first order absorption as shown in equation (1) for a single dose at time t=0. This population-level model describes the concentration time dynamics of the study cohort given dose D of the drug. Here t is time, C(t) is drug concentration over time, Cₘₐₓ is maximum achieved concentration at a particular dose, F is bioavailability, D is dose, V_{d} is volume of distribution, k_{α} is the absorption time constant, and kₑ is the elimination time constant.

$$C ( t ) = \frac{F D {k}_{a}}{{V}_{d} ( {k}_{a} - {k}_{e} )} ( {e}^{- {k}_{e} t} - {e}^{- {k}_{a} t} ) \qquad ( 1 )$$

From the clinical study documentation, for each dose cohort t_{1/2}, tₘₐₓ, and Cₘₐₓ could be established. For example, assuming dose-proportionality, half-life t_{1/2} was estimated for both drugs as the mean over of t_{1/2} over the different single-dose cohorts, as described in the study publications. We could then determine a working value for the elimination time constant kₑ in equation (2):

$${k}_{e} = \frac{ln ( 2 )}{{t}_{1 \text{/} 2}} . \qquad ( 2 )$$

From equation (1), the time of maximum concentration tₘₐₓ is then available from (3):

$${t}_{max} = \frac{ln ( \frac{{k}_{a}}{{k}_{e}} )}{( {k}_{a} - {k}_{e} )} . \qquad ( 3 )$$

With dose-proportionality, tₘₐₓ is independent of dose and is again obtained from the mean over the different single-dose cohorts. The absorption time constant k_{α} is established by identifying the k_{α} which results in a zero error e=0:

$$e ( {k}_{a} ) = {t}_{max} ( {k}_{a} - {k}_{e} ) - ln ( \frac{{k}_{a}}{{k}_{e}} ) = 0. \qquad ( 4 )$$

This is accomplished by graphing e(k_{α}) over a dense grid in k_{α}, identifying a region around the zero-crossing in which e(.) is monotonic in k₀, and then using the interpolation function to estimate the k_{α} with e(k_{α})=0. With the assumption of dose proportionality, we estimated the mean of Cₘₐₓ/D over the single-dose cohorts and defined the constant K

$$K = \frac{F {k}_{a}}{{V}_{d} ( {k}_{a} - {k}_{e} )} . \qquad ( 5 )$$

Then from equation (1) and the definition of Cₘₐₓ,

$$\begin{matrix}K = \frac{( \frac{{C}_{max}}{D} )}{( {e}^{- {k}_{e} {t}_{max}} - {e}^{- {k}_{a} {t}_{max}} )} \\ and & ( 6 ) \\ C ( t ) = K D ( {e}^{- {k}_{e} t} - {e}^{- {k}_{a} t} ) . & ( 7 )\end{matrix}$$

For multiple doses at times τᵢ, we then assume linearity and hav

C(t)=Σ_{τ}_{ᵢ}_{≤t}KD(e^{-k}^{ₑ}^{(t-τ}^{ᵢ}⁾−e^{-k}^{_{α}}^{(t-τ}^{ᵢ}⁾). (8)

All equations were implemented directly in R version 3.2.1 (2015 Jun. 18, see https://cran.r-project.org/).

### Pharmacokinetic Modelling for AQW051

From parameter estimates, we determined the mean over cohorts of tₘₐₓ, t_{1/2}, and Cₘₐₓ/Dose, as shown in Table 1. We then applied the method in equations (1-7) to estimate the model parameters in equation (7), listed in Table 2. FIG. 1 shows the resulting Day 1 concentration profile, while FIG. 2 shows the resulting concentration profile as the PK approaches steady state at Day 14. Note that the IC₅₀=11.77 nM for efficacy (EC₅₀=−40 nM, m.w.=294.398 g). A daily dose of 10 mg provides a concentration near IC₅₀ throughout the 24 hour period. Dose and dosing regime selection for AZD0328 is targeted to achieve similar levels of α7 nAChR agonism to those reported to deliver clinical improvement in MCI with AQW051 (Trenkwalder et al., ibid).

TABLE 1
Model parameters for AQW051-cohort means

| | | | | | | | mean |
|---|---|---|---|---|---|---|---|
| | parameter geometric mean in each cohort | | | | | | over |
| AQW051 | Cohort 1 | Cohort 2 | Cohort 3 | Cohort 4 | Cohort 5 | Cohort 6 | cohorts |
| Dose (mg) | 0.5 | 2.5 | 7.5 | 25 | 75 | 200 | — |
| tₘₐₓ (h) | 8 | 8 | 7.98 | 8 | 4.04 | 4.77 | 6.80 |
| t_{1/2} (h) | — | — | 22.0 | 22.7 | 18.8 | 20.0 | 20.9 |
| Cₘₐₓ (ng/mL) | 0.266 | 1.20 | 4.34 | 20.6 | 47.0 | 162 | — |
| Cₘₐₓ/Dose | 0.53 | 0.48 | 0.58 | 0.82 | 0.63 | 0.81 | 0.64 |
| (ng/mL/mg) | | | | | | | |

TABLE 2
Model parameters for AQW051

| AQW051 | | | K (equation 7 and 8) |
|---|---|---|---|
| parameter | kₑ (h⁻¹) | kₐ (h⁻¹) | (ng/mL/mg) |
| estimate | 0.033 | 0.40 | 0.88 |

### Pharmacokinetic Modelling for AZD0328

From parameter estimates, we determined the mean over cohorts of tₘₐₓ, t_{1/2}, and Cₘₐₓ/Dose, as shown in Table 3. Equations (1-7) are then applied to establish the model parameters listed in Table 4. FIG. 3 shows the resulting Day 1 concentration profile from a single dose, while FIG. 4 shows the resulting concentration file as the PK approaches steady state at Day 14 for once daily dosing. FIG. 5 shows the Day 14 concentration profile for twice-daily dosing. Note that Kᵢ=5 nM for efficacy and Kᵢ=25 nM for toxicity as indicated in these figures.

TABLE 3
Model parameters for AZD0328 - cohort means

| | | | | | | | | mean |
|---|---|---|---|---|---|---|---|---|
| | parameter geometric mean in each cohort | | | | | | | over |
| AZD0328 | | Cohort 2 | Cohort 3 | Cohort 4 | Cohort 5 | Cohort 6 | Cohort 7 | cohorts |
| Dose (mg) | 0.005 | 0.025 | 0.075 | 0.23 | 0.68 | 1.35 | 2 | — |
| tₘₐₓ (h) | 0.8 | 1.44 | 1.59 | 1.38 | 2.08 | 2.95 | 2.45 | 6.21 |
| t_{1/2} (h) | — | 6.25 | 5.94 | 6.69 | 5.98 | 6.87 | 5.6 | 1.81 |
| Cₘₐₓ (nM) | — | 0.63 | 2.08 | 6.37 | 15.04 | 30.98 | 43.88 | — |
| Cₘₐₓ/Dose | — | 25.2 | 27.7 | 27.7 | 22.1 | 22.9 | 21.94 | 24.6 |
| (nM/mg) | | | | | | | | |

TABLE 4
Model parameters for AZD0328

| AZD0328 | | | K (equation 7 and 8) |
|---|---|---|---|
| parameter | kₑ (h⁻¹) | kₐ (h⁻¹) | (nm/mg) |
| estimate | 0.11 | 1.57 | 32.4 |

### Selection of AZD0328 Dose and Dosing Regime

As can be seen in FIGS. 3 and 4, comparatively little accumulation is expected for AZD0328. From FIG. 5, twice daily dosing is seen to provide better coverage over the dosing interval, which more closely matches the pharmacokinetics of the AQW051 dose that delivered an improvement in MCI in the clinic as shown in FIG. 2. A twice-daily dose of 0.3375 mg was thus identified as an appropriate dose to provide drug concentrations above the efficacy Kᵢ for the dosing interval, while maintaining concentrations well below the toxicity Kᵢ. This dose and dosing regime was selected for AZD0328. A twice-daily dos of 0.50 mg or 0.45 mg, provides coverage more than the efficacy Kᵢ for the dosing interval, while maintaining concentrations well below the toxicity Kᵢ. Twice-daily doses in the range of 0.25 mg-0.50 mg provide strong coverage of the efficacy Kᵢ, while maintaining an acceptable safety profile.

Reviewing the clinical data from the Trenkwalder et al. study, we have identified that the plasma level of the α7 agonist needs to at, or above, the reported IC₅₀ value for more than 7 hours, i.e. the 10 mg dose to record an increase in cognitive function, albeit not significantly different from the control group. At the higher dose level, 50 mg, the plasma level of AWQ051 is above the reported IC₅₀ for 24 hours and the improvement in cognitive function was significantly different from the placebo cohort. Hence, our analysis indicates that the plasma level of a α7 agonist needs to be at, or above, the inhibitory concentration for more than 7 hours and sustained as far as possible for 24 hours.

A single dose of AZD0328 even at the highest dose, 0.68 mg, the plasma concentration is at or above the Kᵢ for about 14 hours but at this dose our clinical studies revealed that adverse effects, especially, nausea become apparent. At lower single doses, from 0.25 mg to 0.50 mg, the plasma concentration of AZD03028 is maintained at, or above the Kᵢ, for less than 10 hours following a single dose, but the prevalence of adverse events is markedly reduced. Modelling of twice-daily doses of from 0.25 mg to 0.50 mg reveals that the plasma concentration of AZD0328 is maintained at, or above Kᵢ, for around 20 hours and is thus expected to deliver an therapeutic improvement in MCI and while minimising the likelihood of adverse events occurring. Administration at this dose level and regime is thus expected to deliver clinical benefit, minimise adverse effects and thus be suitable for long term treatment of non-demented Parkinson's disease patients.

We envisage that by dosing AZD0328 maintaining a plasma concentration at or above the Kᵢ for 20 hours the effect on cognition noted in the Trenkwalder publication will be replicated or improved upon while patients undergoing treatment will not suffer from significant treatment side effects. Hence, twice-daily dosing of AZD0328 between 0.25 mg to 0.50 mg per dose will replicate the improvement in cognition.

The dose of AZD0328 to be administered twice daily may be selected from any suitable amount in the range of 0.25 mg to 0.50 mg for example 0.25 mg, 0.30 mg, 0.3375 mg, 0.40 mg, 0.45 mg or 0.50 mg.

AZD0328 is preferably provided for administrations as an oral pharmaceutical composition, for example a tablet or a capsule. For example, AZD0328 may be combined with one or more pharmaceutically acceptable excipients and filled into a two-piece hard shell capsules and or a soft elastic gelatin (SEG) capsule. Alternatively, AZD0328 may be combined with one or more pharmaceutically acceptable excipients and then compressed into a tablet, which may then optionally be coated.

Administration of AZD0328 according to the present description is envisaged on a twice daily basis, i.e. in two individual doses containing between 0.25 mg and 0.50 mg of AZD0328 or a pharmaceutically acceptable salt thereof (wherein a pharmaceutical acceptable salt is used the amount refers to the mass of the basic component (AZD0328) in the salt). The dose is preferable administered every 12 h or as close as possible thereto.

AZD0328 may provided in the form of a kit of parts comprising individual dose units of AZD0328 in the appropriate amount (from 0.25 mg to 0.50 mg) contained in a suitable container such as a blister or bottle and instructions for use of the dose units in the treatment of of mild cognitive impairment in a Parkinson's disease.

Capsules used in the study on which the modelling described above was performed were made by blending AZD0328 tartrate (monotartrate/monohydrate form) with mannitol, povidone and sodium starch glycolate (see Table 5). The blend was the subjected to wet granulation with water as the granulation fluid, then dried before blending with sodium stearyl fumarate. The resultant composition was filled into size 4 hard gelatin capsules (containing gelatin (Ph Eur), titanium dioxide (Ph Eur), and iron oxide black, red and yellow (E172). In embodiments, the compositions for use are capsules according to Table 5.

TABLE 5
Composition of AZD0328 Capsules 0.25 mg

| Component | Quantity per unit (mg) | Function |
|---|---|---|
| AZD0328 Tartrate | 0.445 mg (to give | Drug substance |
| | 0.25 mg as free | |
| | base per capsule) | |
| Mannitol | 47.808 | Filler |
| Povidone | 1.608 | Binder |
| Sodium starch glycolate | 3.217 | Disintegrant |
| (type A) | | |
| Sodium stearyl fumarate | 0.536 | Lubricant |
| Water, purifed^{a} | qs | Granulation Liquid |
| Hard gelatin capsules, | 1 capsule | Capsule |
| brown, size 4 | | |

^{a}Water purified is evaporated

1. (3R)-Spiro[1-azabicyclo[2.2.2]octane-3,2′(3′H)-furo[2,3-b]pyridine, or a pharmaceutically acceptable salt thereof, for use in the treatment or prophylaxis of mild cognitive impairment in Parkinson's disease, wherein the compound is administered in a unit dose of from 0.25 mg to 0.50 mg twice daily.

2. Method of treatment or prophylaxis of mild cognitive impairment in Parkinson's disease, wherein the method involves administration of (3R)-spiro[1-azabicyclo[2.2.2]octane-3,2′(3′H)-furo[2,3-b]pyridine, or a pharmaceutically acceptable salt thereof, to a patient in need thereof at in a unit dose of from 0.25 mg to 0.50 mg twice daily.

3. (3R)-Spiro[1-azabicyclo[2.2.2]octane-3,2′(3′H)-furo[2,3-b]pyridine, or a pharmaceutically acceptable salt thereof, for use in the manufacture of a medicament for the treatment or prophylaxis of mild cognitive impairment in Parkinson's disease, wherein the medicament is to be administered on a twice daily basis and is in a unit dose of from 0.25 mg to 0.50 mg.

4. (3R)-Spiro[1-azabicyclo[2.2.2]octane-3,2′(3′H)-furo[2,3-b]pyridine, or a pharmaceutically acceptable salt thereof, for use in the treatment or prophylaxis of mild cognitive impairment in Parkinson's disease, wherein the compound is dosed in an amount and frequency to maintain a plasma concentration below 25 nM and wherein the plasma concentration is maintained at or above 5 nM for at least 20 h per day.

5. Kit containing (3R)-Spiro[1-azabicyclo[2.2.2]octane-3,2′(3′H)-furo[2,3-b]pyridine, or a pharmaceutically acceptable salt thereof, together with instructions for the use thereof for the treatment or prophylaxis of mild cognitive impairment in Parkinson's disease.
